(19) United States
(12) Plant Patent Application Publication
(10) Pub. No.: US 2016/0360681 P1
(43) Pub. Date: Dec. 8, 2016

(54) Begonia Plant Named `KRBELLE01'

(50) ;

(71) Applicant: Lubbertus H. Koppe, Putten (NL)

(72) Inventor: Lubbertus H. Koppe, Putten (NL)

(73) Assignee: KOPPE ROYALTY B.V., Putten (NL)

(21) Appl. No.: 14/545,699

(22) Filed: Jun. 6, 2015

(51) Int. Cl. A01H 5/00

(52) U.S. Cl. PLT/344

(57) ABSTRACT

A new and distinct cultivar of Begonia plant named ‘KRBELLE01’ characterized by its upright, outwardly spreading and mounded plant habit; moderately vigorous growth habit; dense and bushy appearance; and yellow green-colored leaves with dark brown-colored margins.

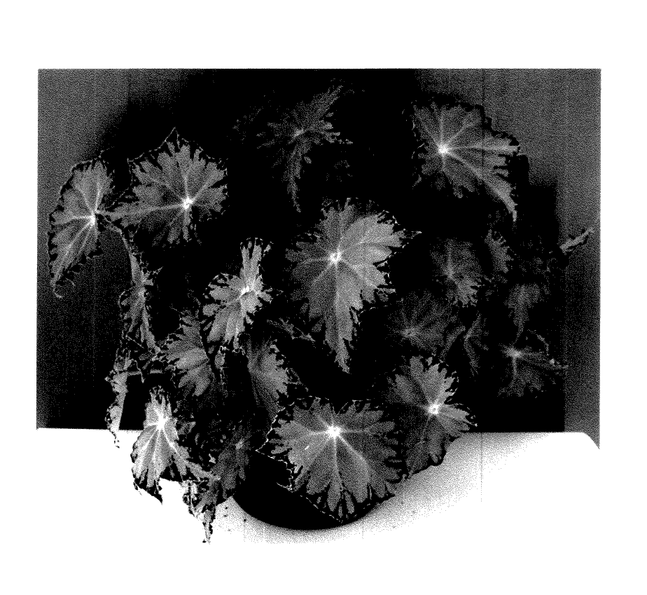

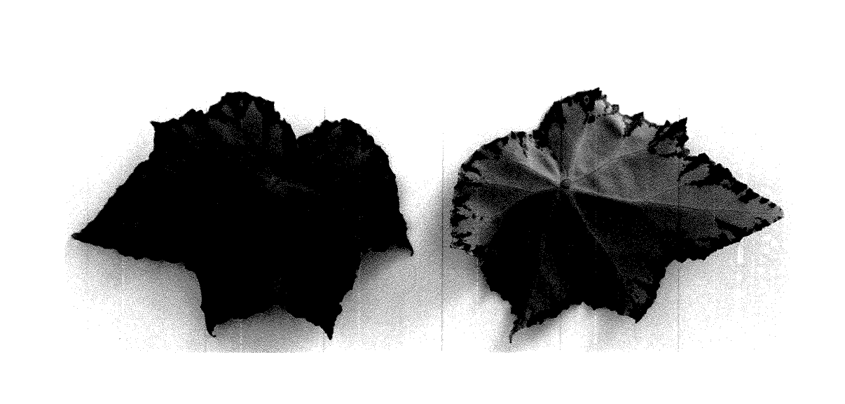

### BOTANICAL DESIGNATION

Begonia hybrida×Begonia bowerae

### CULTIVAR DENOMINATION

‘KRBELLE01’

### BACKGROUND OF THE INVENTION

The present invention relates to a new and distinct cultivar of Begonia plant, botanically known as Begonia hybrida×Begonia bowerae and hereinafter referred to by the name ‘KRBELLE01’.

The new Begonia plant is a product of a planned breeding program conducted by the Inventor in Ermelo, The Netherlands. The objective of the breeding program is to create new types of Begonia plants with unique and attractive foliage.

The new Begonia plant originated from a cross-pollination made by the Inventor in Ermelo, The Netherlands in March, 2004 of Begonia hybrida ‘Cleopatra’, not patented, as the female, or seed, parent with an unnamed proprietary selection of Begonia bowerae, not patented, as the male, or pollen, parent. The new Begonia plant was discovered and selected by the Inventor as a single flowering plant from within the progeny of the stated cross-pollination in a controlled greenhouse environment in Ermelo, The Netherlands in March, 2005.

Asexual reproduction of the new Begonia plant by leaf cuttings taken in a controlled greenhouse environment in Ermelo, The Netherlands since July, 2005 has shown that the unique features of this new Begonia plant are stable and reproduced true to type in successive generations.

### SUMMARY OF THE INVENTION

Plants of the new Begonia have not been observed under all possible combinations of environmental conditions and cultural practices. The phenotype may vary somewhat with variations in environmental conditions such as temperature, daylength and light intensity, without, however, any variance in genotype.

The following traits have been repeatedly observed and are determined to be the unique characteristics of ‘KRBELLE01’. These characteristics in combination distinguish ‘KRBELLE01’ as a new and distinct Begonia plant:
1. Upright, outwardly spreading and mounded plant habit.
2. Moderately vigorous growth habit.
3. Dense and bushy appearance.
4. Yellow green-colored leaves with dark brown-colored margins.

Plants of the new Begonia differ primarily from plants of the female parent, ‘Cleopatra’, in leaf color as plants of ‘Cleopatra’ have dark brown-colored leaves.

Plants of the new Begonia differ primarily from plants of the male parent selection in leaf color as plants of the male parent selection have dark green-colored leaves.

Plants of the new Begonia can be compared to plants of Begonia bowerae ‘Tiger’, not patented. In side-by-side comparisons conducted in Ermelo, The Netherlands, plants of the new Begonia differed primarily from plants of ‘Tiger’ in leaf color as plants of ‘Tiger’ had green-colored leaves with brown-colored spots.

### BRIEF DESCRIPTION OF THE PHOTOGRAPHS

The accompanying colored photographs illustrate the overall appearance of the new Begonia plant showing the colors as true as it is reasonably possible to obtain in colored reproductions of this type. Colors in the photographs may differ slightly from the color values cited in the detailed botanical description which accurately describe the colors of the new Begonia plant. The photograph on the first sheet comprises a side perspective view of a typical plant of ‘KRBELLE01’ grown in a container. The photograph on the second sheet are close up views of the upper (left) and lower (right) surfaces of typical leaves of ‘KRBELLE01’.

### DETAILED BOTANICAL DESCRIPTION

The aforementioned photographs and following observations and measurements describe plants grown during the winter in 13-cm containers in a shaded glass-covered greenhouse in Ermelo, The Netherlands and under cultural practices typical of commercial Begonia production. During the production of the plants, day temperatures averaged 20° C. and night temperatures averaged 18° C. Plants were twelve weeks old when the photographs and description were taken. In the following description, color references are made to The Royal Horticultural Society Colour Chart, 2007 Edition, except where general terms of ordinary dictionary significance are used.
Botanical classification: Begonia hybrida×Begonia bowerae ‘KRBELLE01’.
Parentage:
   Female, or seed, parent.—Begonia hybrida ‘Cleopatra’, not patented.
   Male, or pollen, parent.—Unnamed proprietary selection of Begonia bowerae, not patented.
Propagation:
   Type.—By leaf cuttings.
   Time to initiate roots, summer and winter.—About 20 days at temperatures about 20° C.
   Time to produce a rooted young plant, summer and winter.—About two months at temperatures about 20° C.
   Root description.—Fine, fibrous; white to orange brown in color.
   Rooting habit.—Freely branching; medium density; plants of the new Begonia have not been observed to form tubers.
Plant description:
   Plant habit and form.—Upright, outwardly spreading and mounded plant habit; overall plant shape, flattened globular.
   Growth habit.—Moderately vigorous growth habit and moderate growth rate; suitable for 12-cm and larger containers; under optimal environmental and cultural conditions, usually about ten weeks are required to produce proportional 13-cm potted plants; leaves grow in basal rosettes; dense and bushy appearance, about twelve basal rosettes develop per plant with about 15 leaves per basal rosette; flower development has not been observed on plants of the new Begonia.
   Plant height.—About 22.3 cm.
   Plant width.—About 42.5 cm.
   Leaves.—Arrangement: Alternate, arranged in basal rosettes; simple. Length: About 8.8 cm. Width: About 12.4 cm. Shape: Unequal reniform. Apex: Acuminate. Base: Oblique. Margin: Irregularly dentate to bi-serrate; laciniate. Texture, upper surface: Smooth, glabrous; velvety. Texture, lower surface: Smooth, glabrous; slightly velvety. Luster, upper surface: Matte. Luster, lower surface: Slightly glossy. Venation pattern: Palmate. Color: Developing leaves, upper surface: Close to N144D; margins, close to 183B. Developing leaves, lower surface: Close to N145B; margins, close to 184B. Fully expanded leaves, upper surface: Close to 144A; margins, close to 200A; venation, close to N144B. Fully expanded leaves, lower surface: Close to 139D; margins, close to 187A; venation, close to N144B. Petioles: Length: About 16.6 cm. Diameter: About 5 mm. Texture, upper and lower surfaces: Sparsely pubescent. Glossy, upper and lower surfaces: Moderately glossy. Color, upper and lower surfaces: Close to 145B; lenticels, close to 183B. Stipules: Length: About 1.5 cm. Width: About 8 mm. Shape: Ovate. Apex: Aristate. Margin: Entire. Texture, upper and lower surfaces: Smooth, glabrous. Color, upper and lower surfaces: Close to 145D.
Disease & pest resistance: Resistance to pathogens and pests common to Begonia plants has not been observed on plants of the new Begonia.
Temperature tolerance: Plants of the new Begonia have been observed to tolerate high temperatures of about 35° C. and to be hardy to USDA Hardiness Zone 10.

It is claimed:

1. A new and distinct Begonia plant named ‘KRBELLE01’ as illustrated and described.
